(19) United States
(12) Patent Application Publication
(10) Pub. No.: US 2007/0225616 A1
(43) Pub. Date: Sep. 27, 2007

(54) Wireless urinary incontinence monitoring system

(75) Inventors: William Brown, Fountain Valley, CA (US); Brad Westcott, Fountain Valley, CA (US); Khai Si Luong, Fountain Valley, CA (US); Jeff Sawyer, Fountain Valley, CA (US)

Correspondence Address: Crockett & Crockett, Suite 400, 24012 Calle De La Plata, Laguna Hills, CA, 92653, US

(73) Assignee: Alpine BioMed Corp.

(21) Appl. No.: 11/455,019

(22) Filed: Jun. 15, 2006

Related U.S. Application Data
(60) Provisional application No. 60/691,008, filed on Jun. 15, 2005.

(51) Int. Cl. A61B 5/103; A61B 5/04

(52) U.S. Cl. 600/587; 600/546; 128/903

(57) ABSTRACT

A system and method for ambulatory monitoring of the urinary tract for diagnosis of urinary stress incontinence.

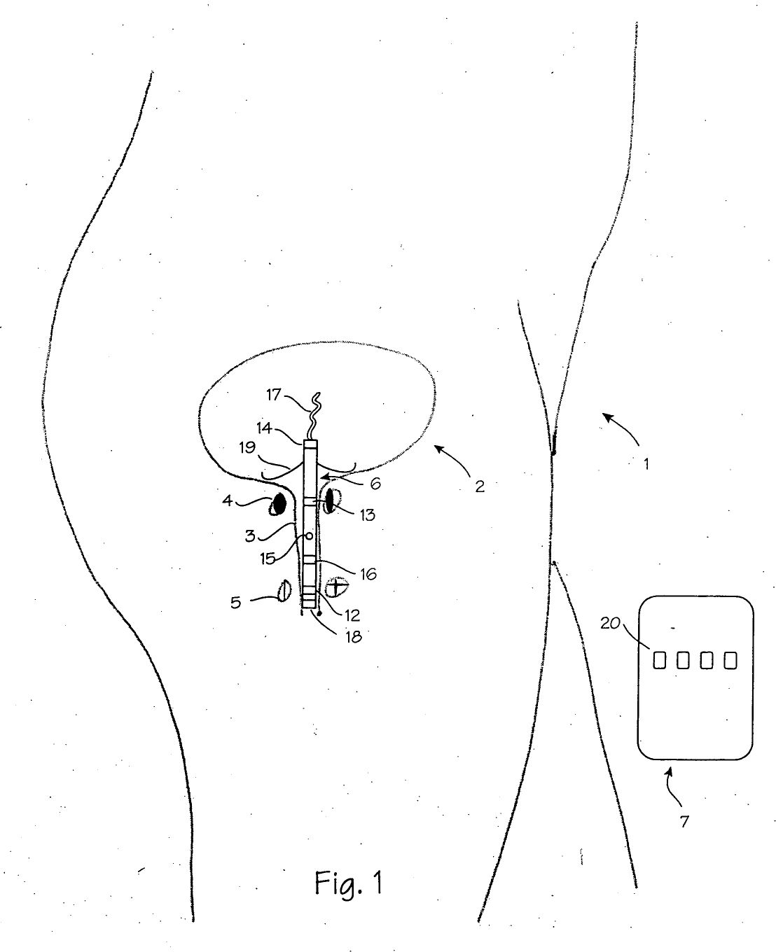

This application claims priority to U.S. Provisional Application 60/691,008, filed Jun. 15, 2005.

### FIELD OF THE INVENTIONS

The inventions described below relate the field of urinary incontinence monitoring.

### BACKGROUND OF THE INVENTION

Urinary incontinence affects approximately 200 million people. Stress urinary incontinence (SUI) is the involuntary loss of urine caused by anatomic defects that allow bladder pressure to be greater than sphincter pressure. SUI is the most prevalent form of incontinence among women and is estimated to affect 30 million women over the age of 18 in the U.S. SUI is a women's condition with 90% of those affected being female. SUI accounts for approximately 50% of incontinent individuals. Urge urinary incontinence (UUI) is the involuntary loss of urine caused by an overactive detrusor muscle. Approximately 20% of those with incontinence have UUI. Mixed urinary incontinence that combines the symptoms of both stress and urge incontinence accounts for 30% of incontinent individuals.

The mainstays of patient evaluation for urinary incontinence are a careful history and physical examination, augmented by a voiding diary and simple office testing (post residual urine volume and cough stress test). Recently, urethral pressure and urinary flow measurements have been used to diagnose the cause of incontinence. Referred to as urodynamics, these tests require that the patient urinate during the course of the test. Approximately 30% of patients cannot reproduce their incontinence symptoms in an office setting. Accordingly, a system designed to permit extended, ambulatory monitoring. Current devices are unsuitable for extended, ambulatory monitoring. Air-perfused catheters and water-perfused catheters require lengthy conduits to air or water sources and pressure transducers. Solid state catheters require wired connections to external devices, making them unsuitable for ambulatory monitoring.

### SUMMARY

The wireless ambulatory urinary incontinence monitoring system employs an indwelling sensor array fitted with several pressure sensors, leak detection sensors, neuromuscular fatigue (NMF) sensors and/or electromyogram (EMG) sensors, and a transmitting system, along with an external receiver for recording signals corresponding to these various sensors which are wirelessly transmitted from the sensor array. Improvement in diagnosis and treatment of urinary incontinence as well as reduction in total treatment costs can be achieved through use of the system.

### BRIEF DESCRIPTION OF THE DRAWINGS

The FIGURE illustrates the ambulatory urinary incontinence monitoring system and the surrounding anatomy of a typical patient.

### DETAILED DESCRIPTION OF THE INVENTIONS

The FIGURE illustrates the ambulatory urinary incontinence monitoring system and the surrounding anatomy of a typical patient 1. The surrounding anatomy includes the bladder 2, the urethra 3, the internal sphincter 4 at the junction of the bladder and the urethra, the external sphincter 5 toward the distal end of the urethra. The indwelling sensor 6 is placed within the urethra such that the various sensors are arranged in relation to the bladder, urethra and sphincters. A receiver 7 is located outside the body, to receive data transmitted from the sensor array, and may be worn or carried by the patient.

The indwelling sensor array 6 is a short, flexible, slender tube or shaft. Several sensors are mounted along the shaft 11, including distal urethral pressure sensor 12 and proximal urethral pressure sensor 13, the bladder pressure sensor 14, the EMG or NMF sensor 15 and the leak detection sensor 16. A wireless transmitter 17 may be located anywhere on the shaft, and, as indicated, is conveniently located at the proximal end of the shaft. A control system is embedded in the sensor array, and is operable to obtain signals from the various sensors and operate the transmitter to transmit data to an external receiver. A battery 18 which supplies power to the wireless transmitter and sensors may be disposed anywhere in the system. The sensor array and its components are sized and dimensioned for placement in the urinary tract and arranged along the shaft such that, when the shaft is placed within the urethra such that the bladder pressure sensor is disposed within the bladder, the proximal urethral pressure sensor may be located within the lumen of the internal sphincter and the distal urethral pressure sensor is located within the lumen of the external sphincter (the device may be provided in numerous sizes to accommodate patients of different size, or one of the urethral pressure sensors may be adjustably relocatable along the shaft prior to insertion). The shaft may be secured within the bladder and urethra with spring clips 19 or other suitable releasable attachment means.

The external receiver 7 is operable to receive and record data transmitted from the sensor array. Any suitable wireless protocol may be used for communication from the sensor array to the receiver. The receiver thus comprises a receiving antenna, a digital storage system sufficient to record received signals and permit access to that data from an associated computer system. The receiver further comprises input means 20 (push buttons, etc.) accessible by the patient which are operable to accept user input to mark events at the discretion of the user. Thus a user can mark the time of any perceived instance of incontinence, leakage or urge to urinate or any other incontinence event.

To use the system, the sensor array is placed within the urethra, with the bladder pressure sensor extending into the bladder. The device is activated and the receiver is held in proximity while the patient partakes in normal daily activity. The system is operated for an extended period as necessary to obtain data pertaining to bladder and urethral function over an ambulatory time period. (By “ambulatory time period” we mean a suitable time period that is sufficiently likely to record events of diagnostic value while the patient is free to move about in normal daily activity. Preferably, the time period is about 24 hours, so that circadian conditions may be observed, and periods of several days may be useful to detect recurrent circadian patterns in incontinence, but this may be varied as necessary according to the patient's tolerance of the sensor and receiver. The patient is instructed to operate the input buttons, during the monitoring and recording of the biological parameter of the urethra and bladder, to mark perceived instances of incontinence, sudden urges to void, etc. After the ambulatory period, the sensor is removed and the data recorded in the receiver is analyzed to diagnose bladder and urethral function.

Doctors may evaluate the data (both the biological parameters and the patient's input regarding perceived events) to determine the cause of urinary incontinence, to evaluate severity of urinary incontinence, to determine appropriate treatment, and to seek patterns of biological parameters that correlate to specific mechanisms of urinary incontinence. By reviewing data collected over an extended time frame, after the patient has escaped the stress of a supervised urination, will enable collection of pertinent data for a large class of patients that cannot void while being observed, catheterized, or otherwise attended by their doctor. By reviewing biological parameter data (such as the pressure measurement data) collected during an ambulatory time period and correlating or comparing this with the patient's marked incontinence events, the absence of urinary incontinence can be confirmed for some patients that perceive urinary incontinence (the perceived symptoms may be further explored to determine the true cause of such perceived incontinence). By reviewing pressure, leakage, and EMG data correlated over ambulatory periods, differential diagnoses may be obtained, such as distinguishing failure of the internal sphincter from mechanical stresses on the bladder, or improper functioning of the nerves controlling the sphincter. These differential diagnoses may not be possible in the typical short-term pressure measurements.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A system for diagnosis the state of the urinary tract of a patient, said system comprising:
a sensor array sized and dimensioned for placement in the urinary tract of the patient such that, when a first portion of the array extends into the bladder of the patient, a second portion of the array resides within the lumen of the internal sphincter of the patient, and a third portion resides within the external sphincter of the patient;
a first pressure sensor disposed on the first portion of the array, a second pressure sensor disposed on the second portion of the array, and a third sensor disposed on the third portion of the array;
a control system and transmitter operably connected to the array, said control system being operable to monitor the output of the sensors and transmit corresponding biological data through the transmitter;
a receiver for receiving and storing data from the control system and transmitter, said receiver being operable to store said data over an ambulatory time period.

2. The system of claim 1 wherein:
an EMG sensor is disposed on the array such that it resides in the urethra when the array is placed in the urinary tract of the patient.

3. The system of claim 1 wherein:
a leak detection sensor is disposed on the array such that it resides in the urethra when the array is placed in the urinary tract of the patient.

4. The system of claim 1 further comprising:
means for recording incontinence events at the discretion of the patient contemporaneously with the recording of biological date.

5. A method of diagnosing the cause of urinary incontinence comprising:
providing a sensor array comprising a first pressure sensor, a second pressure sensor, a third pressure sensor and a means for transmitting pressure measurement data to an external receiver;
providing an external receiver operable to receive the pressure measurement data from the sensor array and record the pressure measurement data;
placing the sensor array within the urinary tract of a patient so that the first sensor array is disposed within the patient's bladder, the second pressure sensor is disposed proximate the patient's internal sphincter, and the third pressure sensor is disposed proximate the patient's external sphincter;
recording pressure measurement data for an ambulatory time period;
analyzing the recorded pressure measurement data in order to diagnose the cause of urinary incontinence.

6. The method of claim 5 further comprising:
providing an EMG sensor on the array and placing the array such that the EMG sensor is placed in operable relationship with the patient's urethra, recording EMG data for the ambulatory time period while also collecting pressure measurement data;
analyzing the recorded EMG data and pressure measurement data in order to diagnose the cause of urinary incontinence.

7. The method of claim 5 further comprising:
providing an leak detection sensor on the array and placing the array such that the leak detection sensor is placed in operable relationship with the patient's urethra, recording leak detection data for the ambulatory time period while also collecting pressure measurement data;
analyzing the recorded leak detection data and pressure measurement data in order to diagnose the cause of urinary incontinence.

8. The method of claim 5 further comprising:
providing the receiver with input means for recording incontinence events as perceived by the patient;
instructing the patient to operate the input means to mark the time or incontinence events during the ambulatory time period;
comparing the recorded pressure measurement data to the patient entered incontinence events to diagnose urinary incontinence.
